(19) United States
(12) Patent Application Publication
(10) Pub. No.: US 2023/0046306 A1
(43) Pub. Date: Feb. 16, 2023

(54) USE OF MESENCHYMAL STEM CELLS AND COMPOSITIONS CONTAINING THEM IN THE MANUFACTURE OF A MEDICAMENT FOR TREATING HARD-TO-HEAL BURN WOUNDS

(71) Applicant: Yan Xie, Ningxia (CN)

(72) Inventors: Yan Xie, Ningxia (CN); Jinli Xiao, Ningxia (CN); Yongzhao Zhu, Ningxia (CN); Nan Xie, Beijing (CN)

(21) Appl. No.: 17/819,052

(22) Filed: Aug. 11, 2022

(30) Foreign Application Priority Data
Aug. 11, 2021 (CN) 202110921024.0

(51) Int. Cl. A61K 35/28; A61K 9/00; A61K 9/06; C12N 5/0775; A61P 17/02

(52) U.S. Cl. CPC A61K 35/28; A61K 9/0019; A61K 9/06; C12N 5/0668; A61P 17/02; C12N 2509/00; C12N 2500/90

(57) ABSTRACT

The invention provides the use of mesenchymal stem cells for preparing drugs/medicines for the treatment of non-healing burn wounds. The inventor found that the mesenchymal stem cells of the present application are used to treat burn wounds and have significant curative effects. They can effectively promote the repair of hard-to-heal burn wounds, increase the healing rate of hard-to-heal burn wounds, and shorten wound healing time, with non-toxic side effects, easy absorption and other advantages. Therefore, the mesenchymal stem cells and the composition containing them can be used to prepare drugs/medicines for treating non-healing burn wounds.

This application claims the benefit of priority to the Chinese patent application No. 202110921024.0, filed before China National Intellectual Property Administration on Aug. 12, 2021, entitled “USE OF MESENCHYMAL STEM CELLS AND COMPOSITIONS CONTAINING THEM IN THE MANUFACTURE OF A MEDICAMENT FOR TREATING HARD-TO-HEAL BURN WOUNDS”, which is incorporated herein in its entirety by reference.

### FIELD OF THE INVENTION

The invention relates to the technical field of new uses of mesenchymal stem cells, in particular to the use of mesenchymal stem cells and compositions containing them for preparing medicines for treating hard-to-heal burn wounds.

### BACKGROUND OF THE INVENTION

Burns are a serious form of trauma, which damages mainly the skin and soft tissues. Burns not only cause structural damage and functional defects of the multi-organ system of the whole body, but also form hard-to-heal wounds, which will bring far-reaching harm to the patient's family and society. Clinically, non-healing burn wounds refer to wounds caused by severe burns that have not healed and have no tendency to heal after one month of treatment. Burn wounds are very different from other types of wounds, such as lacerations, pressure ulcers, venous ulcers, diabetic ulcers, etc. The heat of burns can destroy the body's homeostasis. Most of the hard-to-heal burn wounds have scattered wounds, and tissue edema is common, the patient's immune inflammatory damage is severe, the epithelial growth of the wound edge is stagnant and easy to fall off, causing the wound to expand continuously; and the healed wound has ulcers due to infection, which does not heal for a long time. The pain is often unbearable when changing the dressing of the wound, or the possibility of recurrence after healing is high, and there is a risk of cancer.

There is a big difference between hard-to-heal burn wounds and normal wounds. The hard-healed burn wounds show increased levels of pro-inflammatory cytokines, reactive oxygen radicals and senescent cells, and increased matrix metalloproteinases. Studies have shown that the expressions of PDGF-AB, bFGF, EGF, and TGF-β in chronic wounds are lower than those in acute wounds; the expression of TGF-β1 mRNA can be detected in acute wounds, while the expression of TGF-β1 mRNA in chronic wounds was negative. Studies have shown that the metalloproteinases MMP-2 and MMP-9 in the wound fluid of chronic wounds maintain high levels. Histological in situ zymography showed that the content of urokinase in the granulation tissue of chronic wounds increased, and the level of matrix metalloproteinases increased, which suggests that chronic wounds usually have high proteolytic potential. Yager et al. found that compared with acute wounds, the content of collagenase in chronic wound fluids was significantly higher, and the levels of matrix metalloproteinases and their inhibitors in wound fluids were unbalanced. There are too many activated forms of matrix degrading enzymes in chronic wounds, which hinder the healing of these wounds. Fibroblasts in chronic wounds have reduced migration ability, no response to growth factor signals, and reduced TGF-β receptors and downstream cascade components. In addition, the expression of pro-inflammatory factors such as IL-1β, IL-6, TNF-α and the apoptotic signal factor caspase-3 in chronic wounds are higher than those in acute wounds. The treatment of hard-to-heal burn wounds is a difficult problem in clinical practice, and it is also a research focus and difficulty in the field of burns.

### SUMMARY OF THE INVENTION

In the study, the inventor found that mesenchymal stem cells have excellent therapeutic effect on the burn wound surface, especially the burn hard-to-heal surface, and based on the discovery, the present invention is completed.

The first aspect of the application provides the use of mesenchymal stem cells for preparing medicines for treating hard-to-heal burn wounds.

The second aspect of the present application provides a composition comprising 1×10⁶ to 10×10⁶ mesenchymal stem cells/ml, sodium chloride 0.8-1.0% (w/v) and water for injection.

The third aspect of the present application provides a gel containing mesenchymal stem cells.

The fourth aspect of the application provides the use of the composition of the second aspect of the application or the gel of the third aspect of the application for preparing a medicine for treating hard-to-heal burn wounds.

The fifth aspect of the present application provides the use of a composition containing placental mesenchymal stem cells for the preparation of a medicament for the treatment of hard-to-heal burn wounds, wherein the medicament is an injection, and the composition consists of 1×10⁶ to 10×10⁶/ml Placental mesenchymal stem cells are dispersed in saline.

The inventor found that the mesenchymal stem cells of the present application are used to treat burn wounds and have significant curative effect. They can effectively promote the repair of hard-to-heal burn wounds, increase the healing rate of hard-to-heal burn wounds, shorten wound healing time, with non-toxic side effects, easy absorption and other advantages. Therefore, the mesenchymal stem cells and the composition containing them can be used to prepare medicines for treating non-healing burn wounds.

### DESCRIPTION OF THE DRAWINGS

In order to more clearly illustrate the technical solutions in the embodiments of the present application or the prior art, the following will briefly introduce the drawings that need to be used in the description of the embodiments or the prior art. Obviously, the drawings in the following description are only one embodiment of the present application, and for those of ordinary skill in the art, other embodiments can also be obtained based on these drawings.

FIG. 1. Photomicrograph of the second-generation PMSCs of primary culture.

FIG. 2. Flow cytometry analysis of the surface markers of PMSCs.

FIG. 3. Effects of this invention on rat non-healing burn wounds.

FIG. 4. Quantification of wound healing rate.

FIG. 5. The effect of wound fluid on the proliferation of HK cells and HF cells.

FIG. 6. The effect of wound fluid on the morphology of HK cells and HF cells.

FIG. 7A. PMSCs promote HK migration.

FIG. 7B. PMSCs promote HF migration.

FIG. 8A. Quantification of HK migration.

FIG. 8B. Quantification of HF migration.

FIG. 9A. Quantification of HK proliferation.

FIG. 9B. Quantification of HF proliferation.

### DETAILED DESCRIPTION OF THE INVENTION

The invention is further described with reference to the following drawings and embodiments, so that the objectives, the technical solutions, and the advantages of the present invention are understood more clearly. It is obvious that the embodiments as described are only some embodiments, rather than all embodiments of the present invention. Based on the embodiments of the present invention, all other embodiments obtained by those skilled in the art without creative effort shall fall within the protection scope of the present invention.

On the one hand, the present application provides the use of mesenchymal stem cells for preparing medicines for treating hard-to-heal burn wounds.

On the other hand, the present application provides the use of mesenchymal stem cells for treating hard-to-heal burn wounds.

In some embodiments, the mesenchymal stem cells are selected from at least one of embryonic mesenchymal stem cells, placental mesenchymal stem cells, umbilical cord mesenchymal stem cells, bone marrow mesenchymal stem cells, and adipose mesenchymal stem cells.

The inventors unexpectedly discovered that placental mesenchymal stem cells have a better effect on the treatment of hard-to-heal burn wounds. Furthermore, placental mesenchymal stem cells have a wide range of sources, low allogeneic rejection, and almost no moral and ethical problems. In some preferred embodiments, the mesenchymal stem cells are selected from placental mesenchymal stem cells.

The inventor found in the research that mesenchymal stem cells usually need to be obtained through primary culture. The number of first and second generations of cells is small, which is difficult to meet the demand for dosage. However, if the number of cell passages is too large, such as more than six generations, the cells are prone to aging, which affects cell performance and further affects the therapeutic effect. Therefore, in some embodiments of the present application, the mesenchymal stem cells are mesenchymal stem cells of the third to fifth generation.

The present application does not limit the preparation method of mesenchymal stem cells, and the method of primary culture mesenchymal stem cells in the prior art and conventional cell passaging operations can be used to obtain the mesenchymal stem cells of the present application.

The second aspect of the present application provides a composition comprising 1×10⁶ to 10×10⁶ mesenchymal stem cells/ml, sodium chloride 0.8-1.0% (w/v) and water for injection. The inventor found that at this concentration of mesenchymal stem cells, the composition has a better therapeutic effect on hard-to-heal burn wounds. If the cell concentration is too low, the healing effect on the wound is not obvious. If the cell concentration is too high, the tissue will be over-repaired, such as forming scars.

In some embodiments, the dosage form of the composition is an injection.

In some embodiments, the composition may also include a pharmaceutically acceptable carrier, for example, may contain antioxidants, buffers, bacteriostatic agents, etc.; the injection may be present in a unit-dose or multi-dose container, for example, sealed ampoules and vials.

The third aspect of the present application provides a gel containing mesenchymal stem cells.

The “gel” in this application means a thick liquid or semi-solid preparation made of mesenchymal stem cells and auxiliary materials capable of forming a gel in a suspension or emulsion type. The gel in this application can be used as an external application on the hard-to-heal burn wound. The auxiliary material in the gel may be a pharmaceutically acceptable carrier gel or a bioactive gel, such as ordinary hydrogels, composite hydrogels, degradable hydrogels, bioactive gels, etc. The inventor found in the research that the success of mesenchymal stem cell therapy depends on the effective implantation of living cells into diseased tissues and the realization of the ideal curative effect. The gel is beneficial to protect the mesenchymal stem cells in the hard-to-heal burn wounds, so that they can survive and function.

In some embodiments, the mesenchymal stem cells are selected from at least one of embryonic mesenchymal stem cells, placental mesenchymal stem cells, umbilical cord mesenchymal stem cells, bone marrow mesenchymal stem cells, and adipose mesenchymal stem cells.

In some embodiments, the mesenchymal stem cells are selected from placental mesenchymal stem cells.

In some embodiments, the mesenchymal stem cells are mesenchymal stem cells of the third to fifth generation.

On the other hand, this application also provides a method for treating hard-to-heal burn wounds, including subcutaneous injection of the composition of this application at a distance of 5 mm-10 mm from the edge of the wound. The inventor unexpectedly found that the composition of the present application injected within this range has a more significant effect on treating hard-to-heal burn wounds.

The fourth aspect of the application provides the use of the composition of the second aspect of the application and the gel of the third aspect of the application for preparing a medicine for treating hard-to-heal burn wounds.

The fifth aspect of the present application provides the use of a composition containing placental mesenchymal stem cells for the preparation of a medicament for the treatment of hard-to-heal burn wounds, wherein the medicament is an injection, and the composition consists of 1×10⁶ to 10×10⁶/ml Placental mesenchymal stem cells dispersed in physiological saline.

The inventor unexpectedly discovered in the research that when the placental mesenchymal stem cells are dispersed in physiological saline to prepare an injection for treating hard-to-heal burn wounds, better results can be obtained. Furthermore, the inventors also found that at this concentration of placental mesenchymal stem cells, the composition has a better therapeutic effect on hard-to-heal burn wounds. If the cell concentration is too low, the healing effect on the wound is not obvious. If the cell concentration is too high, the tissue will be over-repaired, such as forming scars.

In some embodiments of the present application, the placental mesenchymal stem cells treat hard-to-heal burn wounds by promoting the migration and/or proliferation of human skin keratinocytes and/or human skin fibroblasts.

In some preferred embodiments, the placental mesenchymal stem cells (PMSCs) are prepared by the following method:

1) In a biological safety cabinet, wash the healthy placental tissue with pre-cooled PBS buffer containing double antibody and gentamicin for more than three times; place the placental tissue on ice and then cut off the placental basement membrane and chorionic membrane (0.5-1.0 cm thickness);

2) Remove the amniotic membrane, cut 0.4-0.6 cm thickness slices of tissue, and place them in a petri dish containing PBS buffer with double antibody;

3) Cut the tissue into pieces of 1-3 mm² respectively, and remove the blood vessels while cutting;

4) Wash the blood in the tissue with PBS buffer; collect the cleaned tissue into a centrifuge tube, add type I collagenase digestion solution, shake on a shaker for 1-3 h at 37° C., stop the reaction with PBS buffer;

5) After mixing, centrifuge at 500 g-550 g for 4-6 min, collect the supernatant; add PBS buffer to the precipitate to make the volume to 20-40 ml, vortex for 25-35 s, and re-suspend the cells;

6) Repeat step (5) until the supernatant is colorless, combine the collected supernatants, centrifuge at 500 g-550 g for 4-6 min, combine the pellets, re-suspend the pellets in serum-free cell culture medium, and sieve with a 100 mesh cell sieve, add serum-free cell culture medium and cultivate overnight;

7) According to the cell growth condition, the medium is changed or refilled. After 5-7 days, the cells are observed to crawl out, which is the first generation of PMSCs;

8) When the cell confluency is greater than 80%, perform routine passaging operations to obtain second to fifth generation PMSCs.

Preferably, after the amniotic membrane is removed, the tissue slices are obtained from the parts with few blood vessels and tight tissue. Those skilled in the art know that the fewer blood vessels, the fewer blood cells, and the more PMSCs in the tight tissue parts.

The inventor found that the PMSCs obtained by the method described above have better effects in treating hard-to-heal burn wounds.

In some embodiments, the PBS buffer containing the double antibody and gentamicin: penicillin 99-110 U/ml, streptomycin 0.08-0.12 mg/ml and gentamicin 0.3-0.35 mg/ml.

In some embodiments, the serum-free cell culture medium includes 3-4 vol % Ultroser G and 0.8-1 vol % GlutaMAX, wherein Ultroser G is a serum substitute and GlutaMAX is a L-glutamine substitute.

The inventors found that the PMSCs cultured with a serum-free cell medium with the above-mentioned component content have better performance in treating hard-to-heal burn wounds.

### Preparation Example 1. Preparation and Identification of PMSCs

Serum-free culture medium: UltraCULTURE™, LONZA, 12-725F, 500 ml; Ultraser G: Pall, 15950-017, 20 ml; GlutaMAX: Gibco, 35050061, 5 ml

Type I collagen digestive fluid: Collagenase Type I, Gibco™, 17018029

Washing buffer: Phosphate buffered saline (PBS) containing 100 U/ml penicillin, 0.1 mg/ml streptomycin and 0.32 mg/ml gentamicin

### Experimental Operation:

1) In a biological safety cabinet, wash the healthy placental tissue with pre-cooled PBS buffer containing double antibody and gentamicin for more than three times; place the placental tissue on ice and then cut off the placental basement membrane and chorionic membrane (0.5-1.0 cm thickness);

2) Remove the amniotic membrane, cut 0.5 cm thickness slices of tissue from the parts with few blood vessels and tight tissue, and place them in a petri dish containing wash buffer;

3) Cut the tissue into pieces of 2 mm² respectively, and remove the blood vessels while cutting;

4) Wash the blood in the tissue with PBS buffer; collect the cleaned tissue into a 50 centrifuge tube, add type I collagenase digestion solution, shake on a shaker for 2 h at 37° C., stop the reaction with PBS buffer, dilute to 50 ml;

5) After mixing, centrifuge at 540 g for 5 min, collect the supernatant; add PBS buffer to the precipitate to make the volume to 30 ml, vortex for 30 s, and re-suspend the cells;

6) Repeat the previous step until the supernatant is colorless, combine the collected supernatants, centrifuge at 540 g for 5 min, combine the pellets, resuspend the pellets in serum-free medium, sieving with 100 mesh cell sieves, and incubate with supplemented medium overnight;

7) According to the cell growth condition, the medium is changed or refilled. After 5-7 days, the cells are observed to crawl out, which is the first generation of PMSCs;

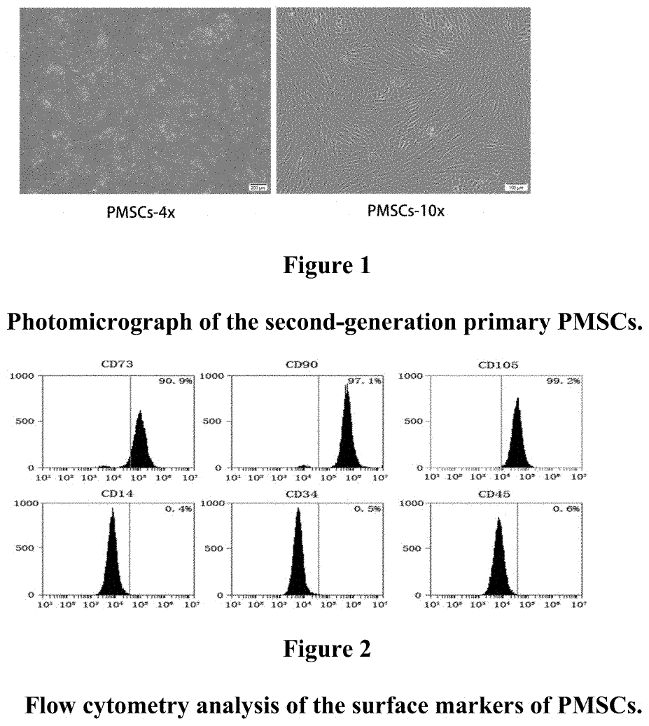

8) When the cell confluency is greater than 80%, perform routine passaging operations to obtain second to fifth generation PMSCs. Among them, the photomicrographs of the second-generation PMSCs under 4× (4×) and 10× (10×) lenses are shown in FIG. 1. It can be seen from FIG. 1 that PMSCs grow in spindle-shaped adherent vortices.

9) PMSCs identification: select the fifth generation of PMSCs, digest and collect the cells in a 15 ml centrifuge tube, rinse twice with PBS, and filter with a sterile 300 mesh cell sieve; after the cell count, adjust the cell density to 1×10⁷/ml. Take 100 μl aliquot into the flow cytometry tube; add 10 ul flow cytometry antibodies: PE-CD73, PE-CD105, PE-IgG1, FITC-CD14, FITC-CD34, FITC-CD45, FITC-CD90 and FITC-HLA-DR. Incubate for 20 min at room temperature in the dark room, shaking once, rinsing twice in PBS, and resuspending the cells in PBS to prepare a 400 μl cell suspension; use flow cytometry for detection. The expression of cell surface markers is shown in FIG. 2. It can be seen from FIG. 2 that the identification marker molecules CD73, CD90, and CD105 are positive, and CD14, CD34, and CD45 are negative, which is consistent with the typical characteristics of the surface marker molecules of mesenchymal stem cells, indicating that the fifth generation cells still retain the characteristics of PMSCs.

### Example 1 PMSCs Promote the Healing of Hard-to-Heal Burn Wounds

In this application, a rat non-healing burn wound model is established by using scalds combined with doxorubicin hydrochloride. The experimental animals used in this study are SPF SD rats (Animal Laboratory of Ningxia Medical University), weighing 280-300 g, female. The experimental operation was approved by the Animal Ethics Committee of Ningxia Medical University (2019-274), and the experimental process complied with the relevant regulations of the “Animal Health and Protection Law”. Doxorubicin hydrochloride (Meilunbio, 25316-40-9) is prepared as a 2 mg/ml solution with normal saline, ready to use. The composition of the present application: the third-generation PMSCs are used and resuspended in physiological saline to obtain the composition of the present application with a PMSCs content of 1×106 pcs/ml. After inducing anesthesia with isoflurane, the SD rats were intraperitoneally injected with 0.5 ml of 3% sodium pentobarbital. After the anesthesia was satisfactory, the rats were depilated on the back. Make two wounds on both sides within 1 cm of the midline of the rat's back in a symmetrical range; use a scalding mold (aluminum block) with a diameter of 2 cm and contact with the wound at 95° C. for 30 seconds. At 6 o'clock, 9 o'clock, 12 o'clock and the wound center, 5 points were injected subcutaneously with 60 ul doxorubicin hydrochloride (2 mg/ml); on the 28th day after the operation, the rats were given the scab on the back of the wound, finally a rat non-healing burn wound model is established.

The effect of PMSCs transplantation on the healing of hard-to-heal burn wounds.

The rats were randomly divided into 3 groups, 3 in each group, and 4 wounds in each rat. The three groups were PMSCs group, recombinant human epidermal growth factor group (GF group) and NS (blank control). The wounds of the rats in the PMSCs group were injected with the composition of the present application at the 3, 6, 9 and 12 points of the wound edge (5 mm away from the outer edge of wounds); and each point was injected subcutaneously with the combination of the present application. The GF group was sprayed with growth factor 4000 IU/10 cm²/wound surface; the NS group was injected with the same amount of normal saline at the corresponding position in the PMSCs group. After the operation is over, cover with sterile gauze and bandage with elastic bandage appropriately. Repeat the above operation every 3 days for a total of 3 times.

### Healing Time

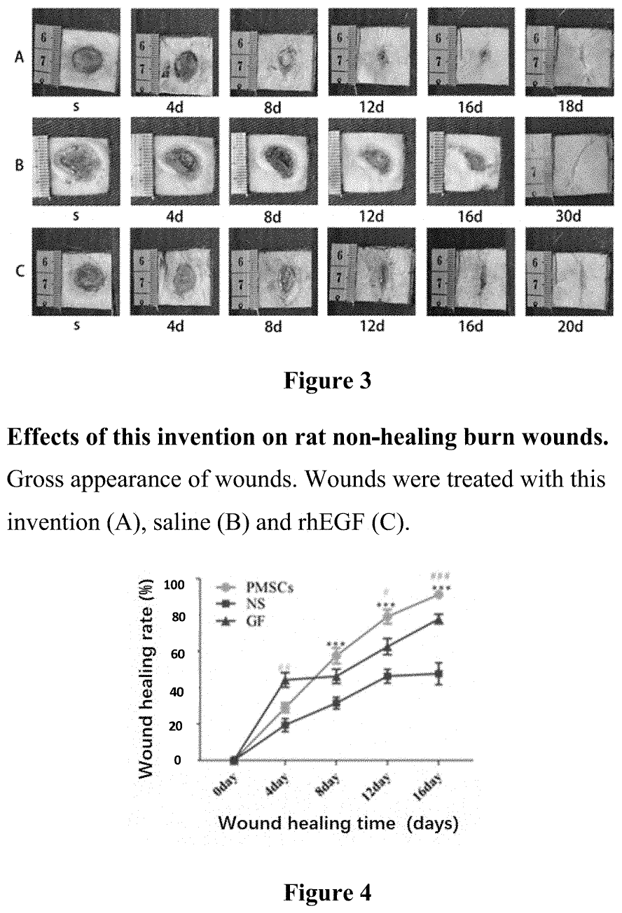

At 0, 4, 8, 12, and 16 days of treatment and when the wound is completely healed (healing rate is 100%), the wound is attached with a ruler and taken with a digital camera to record the wound of the rat. The results are shown in FIG. 3. A represents the PMSCs group, B represents the NS group, and C represents the GF group. It can be seen from FIG. 3 that the wounds in the PMSCs group healed completely in 18 days, the NS group was completely healed in 30 days, and the GF group was completely healed in 20 days. The healing time of PMSCs was significantly shorter than that of the NS group and faster than that of the GF group. In the PMSCs group, there was no obvious redness and swelling during the healing process, and the scab was thin; the wound healed on the 16th day after the scab was lifted (healing rate was greater than 90%). In the NS group, the scab was thick during the healing process, and the epidermis around the wound surface grew slowly to the center; the wounds were relatively large on the 16th day after the scab was lifted. The wounds in the GF group had no obvious infection during the healing process, the scabs were thin, and the wounds remained small on the 16th day after the scabs were lifted.

### Healing Rate

At 0, 4, 8, 12, and 16 days of treatment, the wound is attached with a ruler and taken with a digital camera to record the wound of the rat. The digital image analysis software was used to analyze the wound area and calculate the healing rate. Wound healing rate=(original wound area-unhealed wound area)/original wound area. The data are expressed as mean±standard error. One-way ANOVA and Tukey's post hoc test were used to identify statistically significant differences between the treatment groups, with significance indicated by P<0.05. ImageJ was used to calculate the wound area of mice; GraphPadPrism7 was used to calculate and plot the results. The results are shown in FIG. 4, * means the difference is significant (P<0.05); ** means the difference is extremely significant (P<0.01). It can be seen from FIG. 4 that on 4, 8, 12, and 16 days, in the PMSCs group, the healing rate of hard-to-heal burn wounds gradually increased, which were 28.96%±2.54, 57.63%±4.35, 79.15%±3.85, 91.25%±1.87, respectively. In the NS group, the healing rates of the hard-to-heal burn wounds were 19.43%±3.55, 31.52%±3.19, 46.35%±3.81, 47.72%±5.99, respectively. In the GF group, the healing rates of the hard-to-heal burn wounds were 44.29%±3.96, 46.28%±3.88, 62.47%±4.48, 77.81%±2.62. Compared with the NS group, the wound healing rate of the PMSCs group was significantly improved (P<0.05). Compared with the GF group, the wound healing rate of the PMSCs group was significantly improved (P<0.05). This shows that PMSCs has a better effect on promoting the healing of hard-to-heal burn wounds than external recombinant human epidermal growth factor. More unexpectedly, the inventor found that dispersing placental mesenchymal stem cells in physiological saline has a higher therapeutic effect on hard-to-heal burn wounds than other types of injections, such as compound physiological saline.

### Example 2 the Influence of the Microenvironment of Hard-to-Heal Burn Wounds on Keratinocytes (HK) and Fibroblasts (HF)

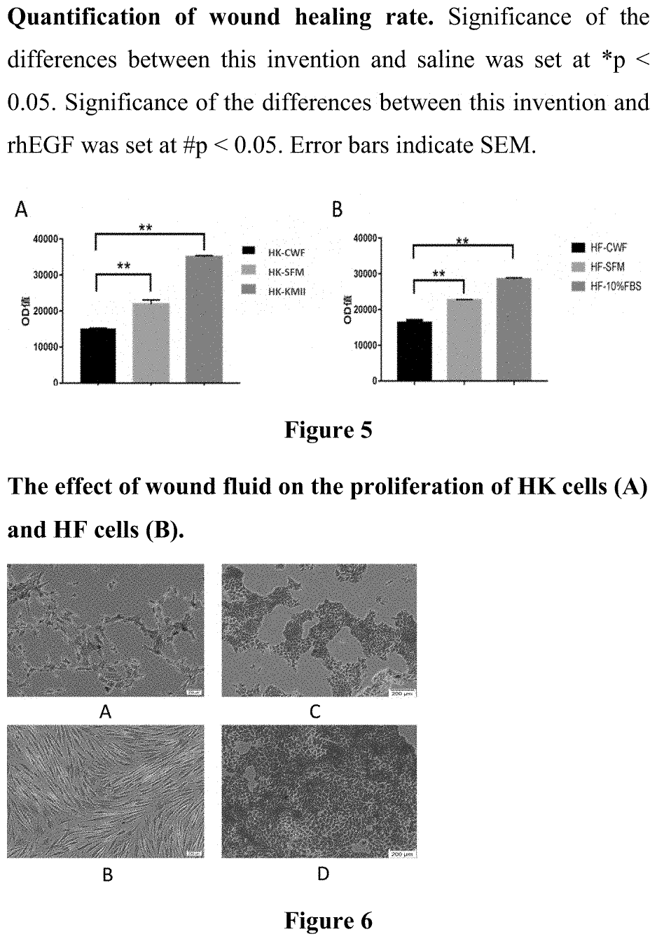

Chronic wound fluids (CWF) were collected from patients with hard-to-heal burn wounds, and stored in a refrigerator at −80° C. HK cells (4×10³ cells/100 ul/well) and HF cells (3×10³ cells/100 ul/well) were seeded in 96-well plates and cultured in DMEM. After 12 hours, DMEM medium was discarded. HF cells were added 100 μL CWF (HF-CWF group), DMEM (HF-SFM group) and DMEM medium (HF-10% FBS group) containing 10% fetal bovine serum (FBS). HK cells were added 100 μL CWF (HK-CWF group), DMEM (HK-SFM group) and KMII medium (EpiLife®, Gibco™, M-EPI-500-CA) (HK-MEM group). After 48 hours of incubation, the cell proliferation was tested with Alamar Blue reagent. The results are shown in FIG. 5. The OD value in the figure reflects the number of cells; the cells were fixed with 4% formalin for 20 minutes and then 0.1% Crystal Violet was counterstained to observe the changes in cell morphology, and the results are shown in FIG. 6.

HF and HK cells proliferate poorly in serum-free DMEM medium (negative control); while they have strong proliferation ability in DMEM containing 10% FBS and KMII medium (positive control). It can be seen from FIG. 5 that after CWF treatment, the cell concentration of HK cells was lower than the negative control group (HK-SFM) by 30.7% (P<0.01), and lower than the positive control group (HK-MEM) by 56.6% (P<0.01) (FIG. 5A). After CWF treatment, the cell concentration of HF cells was lower than the negative control group (HF-SFM) by 27.8% (P<0.01) and lower than the positive control group (HF-10% FBS) by 42.6% (P<0.01) (FIG. 5B). It shows that CWF inhibits the proliferation of keratinocytes and fibroblasts. As can be seen from FIG. 6, after adding CWF, the HF spindle pattern disappears, with no helix (FIG. 6A), and the number of cells decreases compared to normal cells (FIG. 6B). After adding CWF, HK is paved stone form, with small flakes scattered in distribution (FIG. 6C), and the number of cells decreases compared to normal cells (FIG. 6D). It is shown that CWF inhibits the growth of HK and HF.

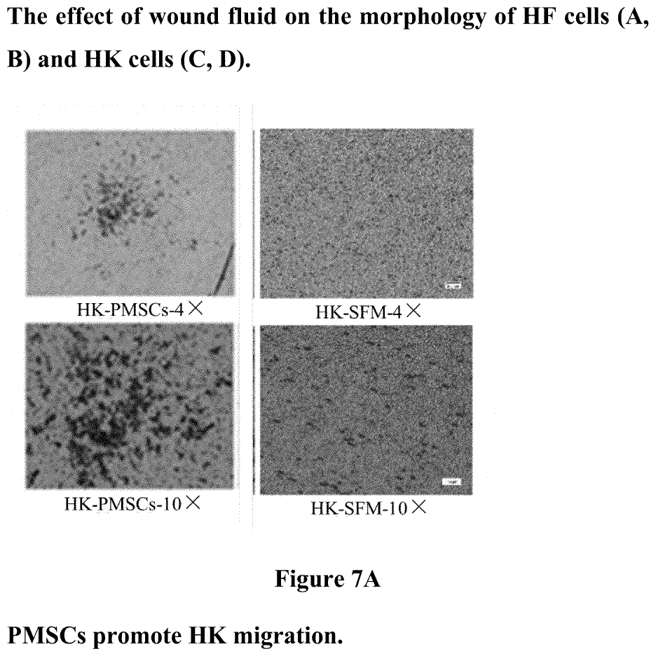

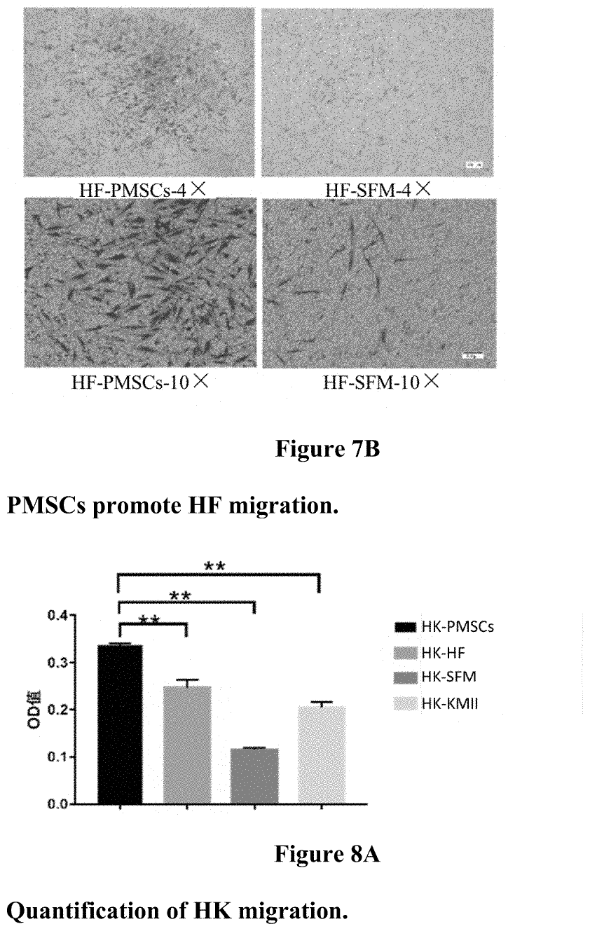

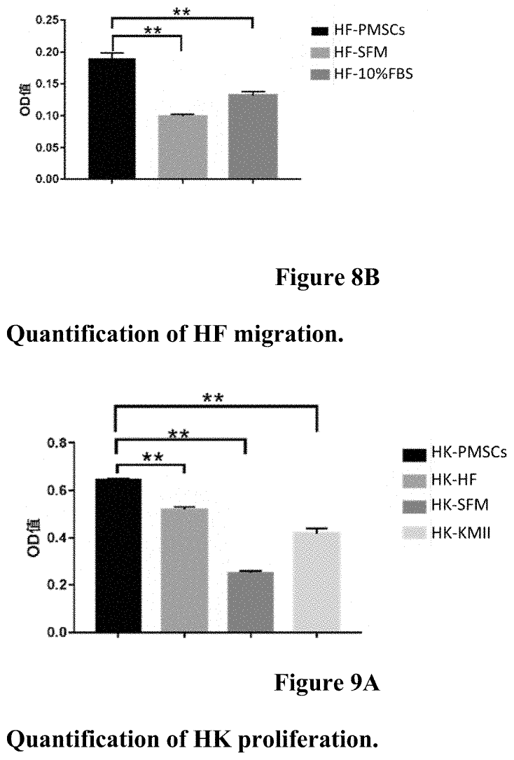

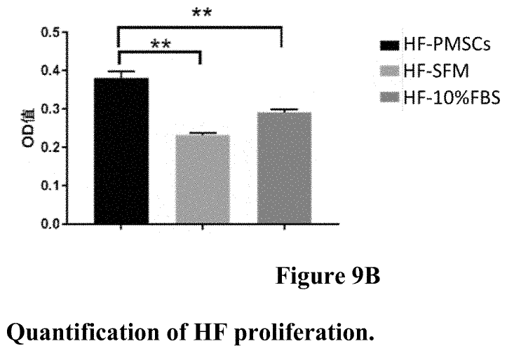

Example 3 The effect of PMSCs on the migration and proliferation of HK and HF After 24 hours, the DMEM in the lower compartment of HF cells was replaced with DMEM (HF-SFM group), 6×10⁴ PMSCs (cultured in serum-free cell culture medium, HF-PMSCs group) and 10% FBS DMEM medium (HF-10% FBS group), DMEM in the lower compartment of HK cells was replaced with 2×10⁴ HF cells (10% FBS DMEM medium cultured, HK-HF group), DMEM (HK-SFM group), 6×10⁴ PMSCs (cultured in serum-free cell culture medium, HK-PMSCs group) and KMII medium (HK-KMII group). After grouping and incubating for 12 hours, some samples of each treatment group were fixed with 4% formalin for 20 minutes and then counterstained with 0.1% crystal violet to observe the cell migration. The microplate reader detects the absorbance OD value of crystal violet staining with Quantitatively reflect the number of migrating cells. The staining results are shown in FIGS. 7A and 7B, and the quantitative results are shown in FIGS. 8A and 8B. In the figure, the absorbance OD value reflects the number of cells. The other samples were incubated for 36 hours (48 hours in total). Alamar Blue reagent was used to detect the total amount of cells above and below the Transwell membrane to reflect the proliferation of HK and HF cells. The results are shown in FIGS. 9A and 9B. OD value reflects the number of cells.

HK cells (4×10⁴ cells/well) and HF cells (2×10⁴ cells/well) were seeded in different upper chambers filled with DMED of Transwells, respectively. 12 h later, DMEM medium was replaced with 3004 CWF, and 600 μL DMEM was added to the corresponding lower chamber. After 24 hours, the DMEM in the lower chamber of HF cells was replaced with DMEM (HF-SFM group), 6×104 PMSCs (cultured in serum-free cell culture medium, HF-PMSCs group) and DMEM containing 10% FBS (HF-10% FBS group); while the DMEM in the lower chamber of HK cells was replaced with 2×10⁴ HF cells (cultured in DMEM containing 10% FBS, HK-HF group), DMEM (HK-SFM group), 6×10⁴ PMSCs (cultured in serum-free cell culture medium, HK-PMSCs group), and KMII medium (HK-KMII group). After incubating for 12 hours, samples were fixed with 4% formalin for 20 minutes and then counterstained with 0.1% crystal violet to observe the cell migration. The microplate reader detects the absorbance OD value after crystal violet staining to quantitatively reflect the number of migrated cells. The staining results are shown in FIGS. 7A and 7B, and the quantitative results are shown in FIGS. 8A and 8B. In the figure, the absorbance OD value reflects the number of cells. The other samples were incubated for another 36 hours (48 hours in total). Alamar Blue reagent was used to detect the total amount of cells above and below the Transwell membrane to reflect the proliferation of HK and HF cells. The results are shown in FIGS. 9A and 9B. OD value reflects the number of cells.

The first column of FIG. 7A is the staining of migrating keratinocytes in the HK-PMSCs group, and the second column is the staining of migrating keratinocytes in the HK-SFM group; the first column of FIG. 7B is the staining of migrating HF cells in the HF-PMSCs group As a result, the second column is the staining of migrating HF cells in the HF-SFM group; it can be seen that after co-cultivation with PMSCs, the migrating number of HK and HF increased significantly, indicating that PMSCs can promote burn wound healing in the microenvironment of HK And the migration of HF, 4× and 10× in the figure represent different magnifications.

The quantitative results of the migration of HK and HF cells are shown in FIGS. 8A and 8B. It can be seen from FIG. 8A that the number of migrated cells in the HK-PMSCs group was 36.9% higher than that in the HK-HF group (P<0.01) and 192.2% in the HK-SFM group. (P<0.01) and 64.6% in the HK-KMII group (P<0.01), indicating that PMSCs can promote the migration of HK in the pathological microenvironment; as can be seen from FIG. 8B, the number of migrating cells in the HF-PMSCs group was higher than that in the HF-SFM group. 91.8% (P<0.01) and 45.4% in the HF-10% FBS group (P<0.01), indicating that PMSCs can promote the migration of HF in the pathological microenvironment.

The quantitative results of the proliferation of HK and HF cells are shown in FIGS. 9A and 9B. FIG. 9A shows that the number of cells in the HK-PMSCs group was 23.3% higher in the HK-HF group (P<0.01), 157.4% in the HK-SFM group (P<0.01) and 53.5% in the HK-KMII group (P<0.01); FIG. 9B shows that the number of cells in the HF-PMSCs group was 65.5% higher than that in the HF-SFM group (P<0.01) and 31% in the HF-10% FBS group (P<0.01). It can be seen that PMSCs can promote the proliferation of HF and HK cells in the pathological microenvironment.

Taken together, the above results show that PMSCs can promote the proliferation and migration of HK and HF cells in the environment of the wound, thus being able to treat the hard-to-burn surface.

The foregoing descriptions are only preferred embodiments of the present application, and are not used to limit the protection scope of the present application. Any modification, equivalent replacement, improvement, etc. made within the spirit and principle of this application are all included in the protection scope of this application.

What claimed is:

1. A composition comprising mesenchymal stem cells at a concentration of 1×10⁶-10×10⁶ cells/ml, sodium chloride at a concentration of 0.8-1.0% (w/v), and water for injection.

2. The composition according to claim 1, wherein the composition is formulated into an injection.

3. The composition according to claim 2, wherein, the mesenchymal stem cells are selected from the group consisting of embryonic mesenchymal stem cells, placental mesenchymal stem cells, umbilical cord mesenchymal stem cells, bone marrow mesenchymal stem cells, and adipose mesenchymal stem cells, and a combination thereof.

4. The composition according to claim 2, wherein, the mesenchymal stem cells are mesenchymal stem cells of third to fifth generations.

5. A gel comprising mesenchymal stem cells.

6. The gel according to claim 5, wherein, the mesenchymal stem cells are selected from the group consisting of embryonic mesenchymal stem cells, placental mesenchymal stem cells, umbilical cord mesenchymal stem cells, bone marrow mesenchymal stem cells, and adipose mesenchymal stem cells, and a combination thereof.

7. The gel according to claim 5, wherein, the mesenchymal stem cells are mesenchymal stem cells of third to fifth generations.

8. A method for treating hard-to-heal burn wounds, comprising administering mesenchymal stem cells to a subject in need thereof.

9. The method according to claim 8, wherein, the mesenchymal stem cells are selected from the group consisting of embryonic mesenchymal stem cells, placental mesenchymal stem cells, umbilical cord mesenchymal stem cells, bone marrow mesenchymal stem cells, and adipose mesenchymal stem cells, and a combination thereof.

10. The method according to claim 8, wherein, the mesenchymal stem cells are mesenchymal stem cells of third to fifth generations.

11. A method for treating hard-to-heal burn wounds, comprising administering the composition according to claim 1 to a subject in need thereof.

12. The method according to claim 11, wherein the mesenchymal stem cells are placental mesenchymal stem cells, and the composition is formulated into an injection.

13. The method according to claim 12, comprising subcutaneously injecting the composition at a distance of 5 mm-10 mm from the edge of the wound.

14. The method according to claim 12, wherein the placental mesenchymal stem cells are placental mesenchymal stem cells of third to fifth generations.

15. The method according to claim 12, wherein the placental mesenchymal stem cells is used to treat hard-to-heal burn wounds by promoting the migration and/or proliferation of human skin keratinocytes and/or human skin fibroblasts.

16. The method according to claim 12, wherein the placental mesenchymal stem cells are prepared by a process comprising the following steps:
1) washing healthy placental tissue in a biological safety cabinet with pre-cooled PBS buffer containing double antibody and gentamicin for more than three times to remove erythrocytes, and placing the placental tissue on ice and then cutting the placental basement membrane and chorionic membrane having a thickness of 0.5-1.0 cm from the placental tissue;
2) removing the amniotic membrane, cutting the obtained tissue into 0.4-0.6 cm thick slices, and placing them in a petri dish containing PBS buffer with double antibody;
3) cutting the slices into pieces each having an area of 1-3 mm², and removing the blood vessels while cutting;
4) washing off the blood in the tissue with PBS buffer, collecting the washed tissue into a centrifuge tube, adding type I collagenase digestion solution, shaking it on a shaker for 1-3 h at 37° C., and stopping the reaction with PBS buffer;
5) after mixing well, centrifuging the centrifuge tube at 500-550 g for 4-6 min, collecting the supernatant; adding PBS buffer to the precipitate until the volume ranges from 20 to 40 ml, vortexing it for 25-35 s, and re-suspending the cells;
6) repeating step 5) until the supernatant is colorless, combining the collected supernatants, centrifuging at 500-550 g for 4-6 min, combining the precipitates, re-suspending the precipitates in a serum-free cell culture medium, sieving with a 100 mesh cell sieve, refilling serum-free cell culture medium, and cultivating the cells overnight;
7) changing or refilling the medium according to the cell growth condition for 5-7 days, and obtaining the cells that are observed to crawl out, which is the placental mesenchymal stem cells of first generation; and
8) performing routine passaging operations when the cell confluency is greater than 80% to obtain placental mesenchymal stem cells of second to fifth generations.

17. The method according to claim 16, wherein the serum-free cell culture medium comprises 3-4 vol. % of Ultroser G and 0.8-1 vol. % of GlutaMAX.

18. A method for treating hard-to-heal burn wounds, comprising administering the gel according to claim 5 to a subject in need thereof.
